(19) United States
(12) Patent Application Publication
(10) Pub. No.: US 2025/0170142 A1
(43) Pub. Date: May 29, 2025

(54) METHODS OF TREATMENT

(71) Applicant: Arena Pharmaceuticals, Ins., New York, NY (US)

(72) Inventors: Dolly PARASRAMPURIA, Dresher, PA (US); Chadwick J. OREVILLO, Lebanon, NJ (US); Philip PERERA, San Tan Valley, AZ (US)

(21) Appl. No.: 18/765,689

(22) Filed: Jul. 8, 2024

Related U.S. Application Data
(63) Continuation of application No. 18/119,321, filed on Mar. 9, 2023, now abandoned.
(60) Provisional application No. 63/318,670, filed on Mar. 10, 2022.

(51) Int. Cl. A61K 31/5517

(52) U.S. Cl. CPC A61K 31/5517

(57) ABSTRACT

Provided is a method of treatment of a 5-hydroxytryptamine (HT)_{2C} receptor-associated disorder comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof.

The developmental and epileptic encephalopathies (DEE) are a heterogeneous group of rare neurodevelopmental disorders. They are characterized by early-onset seizures that are often intractable and are associated with electroencephalographic abnormalities, developmental delay or regression that can worsen over time, and in some cases, early death. These disorders are generally diagnosed in childhood and adolescence; they vary in their etiologies, seizure types, electroencephalographic patterns, cognitive deficits, and prognosis. The International League Against Epilepsy recently expanded this definition to include disorders that may result in developmental delay before epilepsy onset and used the term of DEE to encompass this broader population.

5-HT2 receptor agonists have been shown to be efficacious treatments for a variety of motor seizures and seizure disorders. Specifically, low dose fenfluramine (Fintepla®), a mixed 5-HT2C, 5-HT2B, and 5-HT2A receptor agonist, has recently been approved for the treatment of Dravet syndrome (Food and Drug Administration [FDA] approved label June 2020), and it was shown to be superior to placebo in reducing the frequency of drop seizures in patients with Lennox-Gastaut Syndrome in Phase 3 studies. However, Fintepla® received a Boxed Warning requiring cardiac monitoring because of the association between serotonergic drugs with 5-HT2B receptor agonist activity and valvular heart disease.

There is a significant unmet need for safe and effective treatment for DDE. There also remains a need for alternative compounds for the treatment of diseases and disorders related to the 5-HT_{2C} receptor. The compounds described herein are 5-HT_{2C} receptor agonists that satisfy this need and provide related advantages as well. The present disclosure satisfies this need and provides related advantages as well.

### SUMMARY

Provided is a method of treatment of a 5-hydroxytryptamine (HT)_{2C} receptor-associated disorder comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

Also provided is a method for treating epilepsy comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

Also provided is a method for reducing severity of an epileptic seizure comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

Also provided is a method for treating a seizure disorder comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

Also provided is a method of treating developmental and epileptic encephalopathy (DEE) and other refractory epilepsies comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

These and other aspects of the invention disclosed herein will be set forth in greater detail as the patent disclosure proceeds.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)-N-(2,2-difluoroethyl)-7-methyl-1,2,3,4,6,7-hexahydro-[1,4]diazepino[6,7,1-hi]indole-8-carboxamide.

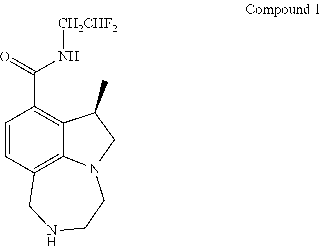

Compound 1, or a pharmaceutically acceptable salt, hydrate, or solvate thereof, is a potent and selective 5-hydroxytryptamine (HT)_{2C} receptor agonist and exhibits increased selectivity for the ligand binding site of 5-HT_{2C} receptors versus those of 5-HT_{2A} and 5-HT_{2B}. Compound 1 displays a binding affinity of 44 nM at the human 5-HT_{2C} receptor and shows >227-fold selectivity for the 5-HT_{2C} receptor versus 5-HT_{2A} and 5-HT_{2B} in comparison to previously developed agonists such as Fintepla® (low dose fenfluramine).

Methods of use of Compound 1, or a pharmaceutically acceptable salt, hydrate, or solvate thereof, are disclosed in U.S. Pat. No. 10,392,390, which is incorporated herein by reference in its entirety for all purposes.

CONVULSIVE/MOTOR SEIZURES: As used here, a “convulsive/motor seizure” refers to a tonic-clonic, tonic, tonic-atonic leading to drop, focal motor, epileptic spasms, myoclonic-atonic leading to drop and seizures. Non-convulsive seizures include myoclonic, absence, atypical absence, or atonic seizures and focal seizures without an observable motor component.

CONVULSIVE/MOTOR SEIZURE-FREE DAY: As used herein, a “convulsive/motor seizure-free day” refers to a day for which diary data are available and no convulsive/motor seizures were reported.

DROP SEIZURE: As used herein, the term “drop seizure” refers to a seizure involving the entire body, trunk or head that leads to a fall, injury, slumping in a chair, or head hitting the surface, or could have led to a fall or injury, depending on the position of the subject at the time of the attack or spell.

AGONIST: As used herein, the term “agonist” refers to a moiety that interacts with and activates a receptor, such as the 5-HT_{2C} serotonin receptor, and initiates a physiological or pharmacological response characteristic of that receptor.

ADMINISTERING: As used herein, “administering” means to provide a compound or other therapy, remedy, or treatment such that an individual internalizes a compound.

ORAL or ORALLY: As used herein, “oral” or “orally” refers to administration of a compound or composition to an individual by a route or mode along the alimentary canal. Examples of enteral routes of administration include, without, limitation, oral, as in swallowing solid (e.g., tablet) or liquid (e.g., syrup) forms; sub-lingual (absorption under the tongue); nasojejunal or gastrostomy tubes (into stomach); intraduodenal administration; as well as rectal administration (e.g., suppositories for release and absorption of a compound or composition by in the lower intestinal tract of the alimentary canal).

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

INTOLERANCE: As used herein, “intolerance” means significant toxicities and/or tolerability issues that led to a reduction in dose or discontinuation of the medication. “Intolerance” can be replaced herein with the term “unable to tolerate.”

ADVERSE EVENT: As used herein, an “adverse event” is an untoward medical occurrence that is associated with treatment with Compound, 1, or a pharmaceutically acceptable salt, hydrate, or solvate thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc.) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL: As used herein, “individual” means any human. In some embodiments, a human individual is referred to a “subject” or “patient.”

DOSE: As used herein, “dose” means a quantity of Compound 1, or a pharmaceutically acceptable salt, hydrate, or solvate thereof, given to the individual for treating or preventing the disease or disorder at one specific time.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

PHARMACEUTICAL COMPOSITION: As used herein, “pharmaceutical composition” means a composition comprising at least one active ingredient, such as Compound 1, including but not limited to, salts of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66: 1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It is understood that when the phrase “pharmaceutically acceptable salts, solvates and hydrates” or the phrase “pharmaceutically acceptable salt, solvate, or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1, or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1, or a pharmaceutically acceptable salt, hydrate, or solvate thereof can be separated into two methods; one method reciting prescribing Compound 1, or a pharmaceutically acceptable salt, hydrate, or solvate thereof and the other method reciting administering Compound 1, or a pharmaceutically acceptable salt, hydrate, or solvate thereof. In addition, for example, a method that recites prescribing Compound 1, or a pharmaceutically acceptable salt, hydrate, or solvate thereof and a separate method of the invention reciting administering Compound 1, or a pharmaceutically acceptable salt, hydrate, or solvate thereof can be combined into a single method reciting prescribing and/or administering Compound 1, or a pharmaceutically acceptable salt, hydrate, or solvate thereof.

### Methods

Provided is a method of treatment of a 5-hydroxytryptamine (HT)_{2C} receptor-associated disorder comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

Also provided is a method for treating epilepsy comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

Also provided is a method for reducing severity of an epileptic seizure comprising: prescribing and/or administering to an individual in need thereof, (R)-N-(2,2-difluoroethyl)-7-methyl-1,2,3,4,6,7-hexahydro-[1,4]diazepino [6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

Also provided is a method for treating a seizure disorder comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

In some embodiments, the seizure disorder is selected from epilepsy, epilepsy with generalized tonic-clonic seizures, epilepsy with myoclonic absences, frontal lobe epilepsy, temporal lobe epilepsy, Landau-Kleffher Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

Also provided is a method of treating developmental and epileptic encephalopathy (DEE) and other refractory epilepsies comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

In some embodiments, the DEE is chosen from Lennox-Gastaut syndrome, Dravet syndrome, Doose syndrome (Epilepsy with Myoclonic Atonic Seizures (EM AS)), West syndrome (Infantile Spasms), Landau-Kleffner syndrome, and genetic disorders such as CDKL5 encephalopathy or CHD2 encephalopathy.

In some embodiments, the DEE is chosen from Ohtahara syndrome, Lennox-Gastaut syndrome, Dravet syndrome, Doose syndrome, West syndrome, Landau-Kleffner syndrome, tuberous sclerosis complex, CDKL5 encephalopathy, CHD2 encephalopathy, early myoclonic encephalopathy, epilepsy of infancy with migrating focal seizures, epilepsy with myoclonic-atonic seizures, and epileptic encephalopathy with continuous spike-and-wave during sleep.

In some embodiments, the individual has a comorbid conditions, such as intellectual disability, autism spectrum disorder, and/or behavioral problems.

In some embodiments, the method provides improvement in at least one symptom selected from ataxia, gait impairment, speech impairment, vocalization, impaired cognition, abnormal motor activity, clinical seizure, subclinical seizure, bypotonia, hypertonia, drooling, mouthing behavior, aura, convulsions, repetitive movements, unusual sensations, frequency of seizures and severity of seizures.

In some embodiments, the administration results in an improvement in the frequency of convulsive/motor seizures and other seizure types. In some embodiments, the administration results in an improvement in one or more of the following:
frequency of observed countable motor seizures;
number of total seizures;
frequency of non-convulsive seizure;
number of episodes of status epilepticus;
frequency of use of rescue medication; and/or
number of countable motor seizure-free days.

In some embodiments, the administration results in an improvement in the Subject/Caregiver and Investigator Clinical Global Impression of Improvement (CGI-I), the Investigator Clinical Global Impression of Severity (CGI-S), and/or the 55-item Quality of Life in Childhood Epilepsy Questionnaire (QOLCE-55). In some embodiments, the administration results in at least a 1-point change from baseline in CGI-I and/or CGI-S.

In some embodiments, prior to administration, the individual had treatment resistant countable motor seizures with an average of ≥4 observed/countable motor seizures per 4-week period while on stable ASM treatment.

In some embodiments, the individual has a DEE but does not have Dravet syndrome or Lennox-Gastaut Syndrome.

In some embodiments wherein the individual has a DEE but does not have Dravet syndrome or Lennox-Gastaut Syndrome, the individual had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

In some embodiments, the individual has Dravet syndrome.

In some embodiments wherein the individual has Dravet syndrome, prior to administration, the individual had:
onset of seizures between 3 and 12 months of age in an otherwise healthy infant; a history of seizures that were either generalized tonic-clonic or unilateral clonic or bilateral clonic;
normal initial development; and/or
a history of developmental delay.

In some embodiments wherein the individual has Dravet syndrome, prior to administration, the individual had:
an emergence of another seizure type;
prolonged exposure to warm temperatures-induced seizures and/or seizures that were associated with fevers due to illness or vaccines, hot baths, high levels of activity, and sudden temperature changes, and/or
seizures were induced by strong natural and/or fluorescent lighting.

In some embodiments wherein the individual has Dravet syndrome, prior to administration, the individual had genetic test results consistent with a diagnosis of Dravet syndrome.

In some embodiments. the individual has Lennox-Gastaut Syndrome.

In some embodiments wherein the individual has Lennox-Gastaut Syndrome, prior to administration, the individual had:
a history of tonic seizures or tonic/atonic seizures;
more than 1 type of generalized seizure, including but not limited to generalized tonic-clonic, tonic-atonic, atonic, tonic, myoclonic, or drop seizures;
a history of seizure before 8 years of age.
a history of developmental delay.
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

In some embodiments, the dose of Compound 1, or a pharmaceutically acceptable salt, hydrate, or solvate thereof, is selected from, or from about, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 mg daily. In some embodiments, the dose of Compound 1, or a pharmaceutically acceptable salt, hydrate, or solvate thereof, is selected from, or from about, 9, 12, 15, 18, 21, 24, 27, 30, 33, 36, 39, 42, 45, 48, 51, 54, 57, 60, 63, 66, 69, or 72 mg daily. In some embodiments, the dose of Compound 1, or a pharmaceutically acceptable salt, hydrate, or solvate thereof, is 9 mg daily. In some embodiments, the dose of Compound 1, or a pharmaceutically acceptable salt, hydrate, or solvate thereof, is 18 mg daily. In some embodiments, the dose of Compound 1, or a pharmaceutically acceptable salt, hydrate, or solvate thereof, is 36 mg daily. In some embodiments, the dose of Compound 1, or a pharmaceutically acceptable salt, hydrate, or solvate thereof, is 54 mg daily. In some embodiments, the dose of Compound 1, or a pharmaceutically acceptable salt, hydrate, or solvate thereof, is 72 mg daily.

In some embodiments, the dose of Compound 1, or a pharmaceutically acceptable salt, hydrate, or solvate thereof, is 3 mg TID. In some embodiments, the dose is 3 mg TID. In some embodiments, the dose is 6 mg TID. In some embodiments, the dose is 9 mg TID. In some embodiments, the dose is 12 mg TID. In some embodiments, the dose is 9 mg TID. In some embodiments, the dose is 18 mg TID. In some embodiments, the dose is 9 mg TID. In some embodiments, the dose is 24 mg TID.

In some embodiments, the dose of Compound 1, or a pharmaceutically acceptable salt, hydrate, or solvate thereof, is titrated to 9 mg daily. In some embodiments, the dose of Compound 1, or a pharmaceutically acceptable salt, hydrate, or solvate thereof, is titrated to 18 mg daily. In some embodiments, the dose of Compound 1, or a pharmaceutically acceptable salt, hydrate, or solvate thereof, is titrated to 36 mg daily. In some embodiments, the dose of Compound 1, or a pharmaceutically acceptable salt, hydrate, or solvate thereof, is titrated to 54 mg daily. In some embodiments, the dose of Compound 1, or a pharmaceutically acceptable salt, hydrate, or solvate thereof, is titrated to 72 mg daily.

In some embodiments, the dose of Compound 1, or a pharmaceutically acceptable salt, hydrate, or solvate thereof, is down-titrated to, or to about, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17 mg daily. In some embodiments, the dose of Compound 1, or a pharmaceutically acceptable salt, hydrate, or solvate thereof, is down-titrated to 54 mg daily. In some embodiments, the dose of Compound 1, or a pharmaceutically acceptable salt, hydrate, or solvate thereof, is down-titrated to 36 mg daily. In some embodiments, the dose of Compound 1, or a pharmaceutically acceptable salt, hydrate, or solvate thereof, is down-titrated to 18 mg daily. In some embodiments, the dose of Compound 1, or a pharmaceutically acceptable salt, hydrate, or solvate thereof, is down-titrated to 9 mg daily.

In some embodiments, the increasing the dose of Compound 1, or a pharmaceutically acceptable salt, hydrate, or solvate thereof, in increments of 3 mg TID every 5 days until the optimized dose is administered. In some embodiments, the optimized dose is 3 mg TID. In some embodiments, the optimized dose is 6 mg TID. In some embodiments, the optimized dose is 9 mg TID. In some embodiments, the optimized dose is 12 mg TID.

In some embodiments, the titration scheme comprises prescribing and/or administering Compound 1, or a pharmaceutically acceptable salt, hydrate, or solvate thereof, at an initial dose equivalent to 6 mg of Compound 1 three time daily for about five days and, provided that the individual tolerates the initial dose and that the individual has not had an adequate response, increasing the dose.

In some embodiments, the increased dose is equivalent to 9 mg of Compound 1 three time daily.

In some embodiments, the titration scheme further comprises administering Compound 1, or a pharmaceutically acceptable salt, hydrate, or solvate thereof, at the increased dose for about five days.

In some embodiments, if the individual does not tolerate the increased dose, the optimized dose is the initial dose.

In some embodiments, if the individual tolerates the increased dose and if the individual has had an adequate response, the optimized dose is the increased dose.

In some embodiments, the titration scheme further comprises administering the optimized dose of Compound 1, or a pharmaceutically acceptable salt, hydrate, or solvate thereof, to the individual.

In some embodiments, if the individual tolerates the increased dose and if the individual has not had an adequate response, the method further comprises increasing the dose.

In some embodiments, the further increased dose is equivalent to about 12 mg of Compound 1 three times daily.

In some embodiments, if the individual does not tolerate the further increased dose, the optimized dose is the increased dose.

In some embodiments, if the individual tolerates the further increased dose and if the individual has had an adequate response, the optimized dose is the further increased dose.

In some embodiments, the titration scheme further comprises administering the optimized dose of Compound 1, or a pharmaceutically acceptable salt, hydrate, or solvate thereof, to the individual.

In some embodiments, for example, when the individual does not tolerate the optimized dose of Compound 1, or a pharmaceutically acceptable salt, hydrate, or solvate thereof, the method further comprises a down-titration scheme. In some embodiments, the down-titration scheme comprises reducing the dose of Compound 1, or a pharmaceutically acceptable salt, hydrate, or solvate thereof, being administered to the individual by, or by about, 1, 2, 3, 3, 4, 5, 6, 7, 8, 9, 10, 11, or 12 mg per day. In some embodiments, the down-titration scheme comprises reducing the dose of Compound 1, or a pharmaceutically acceptable salt, hydrate, or solvate thereof, being administered to the individual once. In some embodiments, the down-titration scheme comprises reducing the dose of Compound 1, or a pharmaceutically acceptable salt, hydrate, or solvate thereof, being administered to the individual more than once. In some embodiments, the down-titration scheme comprises reducing the dose of Compound 1, or a pharmaceutically acceptable salt, hydrate, or solvate thereof, being administered to the individual in increments of 3 mg TID every 5 days until the individual is no longer being administered Compound 1, or a pharmaceutically acceptable salt, hydrate, or solvate thereof.

In some embodiments, the dose of Compound 1, or a pharmaceutically acceptable salt, hydrate, or solvate thereof, is down-titrated to address an observed side effect. In some embodiments, the dose of Compound 1, or a pharmaceutically acceptable salt, hydrate, or solvate thereof, is down-titrated to minimize the risk of a withdrawal induced side effect.

In some embodiments, the individual is also being administered an antiepileptic drug or antiseizure medicine. In some embodiments, the individual is also being administered an antiepileptic drug effective in suppressing interictal epileptiform discharges (e.g., benzodiazepines, valproic acid, and lamotrigine). In some embodiments, the individual is also being administered an immunomodulatory therapies (e.g., corticosteroids, intravenous immunoglobulin [IVIG], plasmapheresis). In some embodiments, the individual is also being administered a ketogenic diet.

Also provided are pharmaceutical compositions comprising a standard dose of Compound 1, or, a pharmaceutically acceptable salt, a hydrate or solvate thereof and, optionally, one or more pharmaceutically acceptable carriers. Also provided are pharmaceutical compositions comprising Compound 1, or, a pharmaceutically acceptable salt, a hydrate or solvate thereof, optionally, one or more pharmaceutically acceptable carriers. The carrier(s) must be “acceptable” in the sense of being compatible with the other ingredients of the formulation and not overly deleterious to the recipient thereof.

In some embodiments, Compound 1, or a pharmaceutically acceptable salt, a hydrate or solvate thereof, is administered as a raw or pure chemical, for example as a powder in capsule formulation.

In some embodiments, Compound 1, or a pharmaceutically acceptable salt, a hydrate or solvate thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

In some embodiments, the Compound 1, or a pharmaceutically acceptable salt, hydrate, or solvate thereof, is formulated in a manner suitable for oral administration.

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For oral administration, the pharmaceutical composition may be in the form of suitable for administration via gastrostomy tube or percutaneous endoscopic gastrostomy tube.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### EXAMPLES

### Example 1: Single Ascending Dose Study With Healthy Subjects

A single ascending dose study with healthy subjects was conducted to evaluate the pharmacokinetics (PK), pharmacodynamics (PD), safety and tolerability of single doses of Compound 1 (“Study Drug”) administered to healthy subjects.

The Study Drug is a potent, highly selective 5-HT_{2C} superagonist with low nanomolar binding affinity of 44 nM to the human 5-HT_{2C} receptor and >200-fold selectivity versus 5-HT2a and 5-HT2b. The Study Drug is being developed for the treatment of developmental and epileptic encephalopathies (DEEs).

This was a randomized, double-blind, placebo-controlled, single-ascending dose study in healthy adults, ages 18 to 55 years and body mass index (BMI) of 18.5-30.0 kg/m². Subjects were randomized to receive single doses of powder in capsule formulation of 1, 3, 6, 12 or 24 mg, under fasting conditions. Each cohort consisted of 6 active and 2 placebo subjects. Serial plasma and urine PK, and prolactin samples were collected up to 72 h. Safety was assessed continuously from signing of informed consent form through follow up visit.

Forty female subjects with mean age of 35.0 years and BMI of 24.0 kg/m² were enrolled. Study Drug was rapidly absorbed with peak plasma concentrations occurring between 1 and 1.5 hours after dosing. The elimination half-life was approximately 5 to 7 hours. Peak and total exposure increased with dose, although the increase appears to be slightly greater than dose proportional especially at higher doses. Less than 5% of the administered dose (<5%) was eliminated renally. Thus, metabolism appears to be the major route of clearance.

Mean (SD) of PK parameters following single dose administration under fasting conditions are shown below.

| | 1 mg | | 3 mg | | 6 mg | | 12 mg | | 24 mg | |
|---|---|---|---|---|---|---|---|---|---|---|
| Statistic | N = 6 | | N = 6 | | N = 6 | | N = 6 | | N = 6 | |
| Cₘₐₓ | 1.08 | (0.497) | 6.36 | (2.12) | 9.52 | (5.10) | 17.0 | (5.20) | 59.3 | (25.5) |
| (ng/mL) | | | | | | | | | | |
| Tₘₐₓ (h) | 1.54 | | 1.02 | | 1.50 | | 1.25 | | 1.07 | |
| Median | | | | | | | | | | |
| AUC_{0-inf} | NC | | 40.9 | (19.3) | 58.7 | (34.0) | 101 | (46.0) | 288 | (125) |
| (h*ng/mL) | | | | | | | | | | |
| t_{1/2} (h) | NC | | 4.94 | (1.28) | 6.66 | (2.88) | 4.67 | (1.50) | 5.09 | (0.703) |
| CL/F (L/h) | NC | | 92.4 | (54.1) | 134 | (70.3) | 150 | (87.8) | 93.7 | (30.6) |
| Vz/F (L) | NC | | 608 | (236) | 1230 | (832) | 929 | (430) | 697 | (263) |
| CL_{R} (L/h) | 3.00 | (0.572) | 3.62 | (0.888) | 3.42 | (0.829) | 4.25 | (0.755) | 3.75 | (0.844) |

Prolactin increased within 2 h in a dose-and concentration-dependent manner, with greater than dose proportional increase between 6 mg and 12 mg doses. Although there was high variability and considerable overlap across doses in prolactin levels for subjects on Study Drug, the mean % CFB at 2 hours increased in a dose-dependent manner, with a greater than dose proportional increase between 6 mg and 12 mg doses. By 24-hour postdose, prolactin levels returned to baseline for only the 6 mg dose level. Placebo and 1 mg dose groups showed an initial decline at 2 hours, followed by higher prolactin concentrations at 24 hours, but this could be the result of diurnal variation, together with variability in the data.

The most common treatment emergent adverse events were headache, postural dizziness, and nausea, which were mild to moderate in severity.

In sum, Study Drug was rapidly absorbed into the systemic circulation following administration of a single oral dose of Study Drug PIC formulation. Peak plasma concentrations were observed at a median Tmax range of 1.02 to 1.54 hours after administration of 1 to 24 mg as a single oral dose. Study Drug mean half-life ranged from 4.67 to 6.66 hours across 1 to 24 mg dose groups. Since less than 5% of dose was eliminated by the renal route, a majority of the elimination likely occurred via metabolism.

### Example 2: Multiple Ascending Dose Study With Healthy Subjects

A randomized, double-blind, placebo-controlled, multiple ascending dose study of Study Drug in healthy subjects was conducted to evaluate PK, PD, safety, tolerability of multiple doses of Study Drug.

This study enrolled healthy adults, ages 18 to 55 years and body mass index of 18.5-30.0 kg/m² in two parts. Subjects in one part received thrice daily (every 8 h) doses of powder-in-capsule without titration for 14 days, and in another part received the highest dose with 3-day titration. On PK sampling days, subjects fasted overnight prior to the morning dose. Serial plasma and prolactin samples were collected on Day 1 and Day 14, and urine samples were collected on Day 14. Safety was assessed continuously from signing of informed consent form through follow up visit.

Plasma and urine concentration time data were analyzed using noncompartmental methods to report the following key PK parameters: Cₘₐₓ, Tₘₐₓ, AUCₜₐᵤ, C_{trough}, Cₐᵥ, Vss/F, CL/F, Racc, Cₘₐₓ, Racc, AUC, T_{1/2}. Concentration time data and PK parameters were summarized using descriptive statistics. Trough plasma concentrations were plotted to visually assess the steady state.

Prolactin serum concentrations were summarized by timepoint, dose and part. Absolute and percent change from baseline in prolactin value was calculated, with predose value on Day 1 considered as the baseline value. Observed prolactin data, absolute and percent change from baseline versus time were plotted.

Forty-three (16M, 27F) subjects were enrolled. Study Drug was rapidly absorbed. Significant accumulation occurred upon multiple dosing TID. For doses up to 18 mg TID, steady-state was achieved by Day 10, in most cases. At steady state, about 30% of the dose was eliminated by the renal route in 48 h (in non-titrated groups).

Provided below is a summary of pharmacokinetic parameters by cohort.

| | | No | No | No | No | 3-Day |
|---|---|---|---|---|---|---|
| | | Titration | Titration | Titration | Titration | TItration |
| | | 3 mg | 6 mg | 12 mg | 18 mg | 24 mg |
| Parameter | Statistic | N = 6 | N = 6 | N = 7 | N = 6 | N = 8 |
| Cₘₐₓ | N | 6 | 6 | 6 | 6 | 4 |
| (ng/ml) | Mean | 6.61 | 20.7 | 44.9 | 98.6 | 107 |
| | SD | 2.61 | 7.21 | 15.8 | 49.6 | 46.2 |
| | CV % | 39.5 | 34.8 | 35.1 | 50.3 | 43.1 |
| Tₘₐₓ | N | 6 | 6 | 6 | 6 | 4 |
| (h) | Minimum | 1.00 | 0.50 | 1.00 | 1.00 | 1.00 |
| | Median | 1.01 | 1.78 | 1.27 | 2.00 | 1.04 |
| | Maximum | 1.50 | 2.05 | 1.50 | 2.03 | 2.00 |
| AUCₜₐᵤ (h*ng/mL) | N | 6 | 6 | 6 | 6 | 4 |
| | Mean | 27.1 | 99.8 | 213 | 480 | 480 |
| | SD | 13.4 | 36.5 | 77.7 | 276 | 243 |
| | CV % | 49.4 | 36.6 | 36.5 | 57.4 | 50.5 |
| AUC_{0-inf} | N | 4 | 6 | 6 | 6 | 4 |
| (h*ng/mL) | Mean | 46.1 | 152 | 330 | 775 | 741 |
| | SD | 25.6 | 55.7 | 119 | 421 | 331 |
| | CV % | 55.6 | 36.6 | 36.2 | 54.3 | 44.6 |
| AUC₀₋ₗₐₛₜ | N | 6 | 6 | 6 | 6 | 4 |
| (h*ng/mL) | Mean | 38.6 | 150 | 327 | 773 | 737 |
| | SD | 20.4 | 56.1 | 118 | 421 | 332 |
| | CV% | 52.8 | 37.3 | 36.1 | 54.4 | 45.1 |
| T1/2 | N | 4 | 6 | 6 | 6 | 4 |
| (h) | Mean | 5.56 | 4.81 | 5.96 | 5.64 | 6.50 |
| | SD | 0.790 | 0.730 | 1.34 | 1.01 | 2.76 |
| | CV % | 14.2 | 15.2 | 22.4 | 18.0 | 42.4 |
| C_{trough} | N | 6 | 6 | 6 | 6 | 4 |
| (ng/mL) | Mean | 1.94 | 7.02 | 15.8 | 37.7 | 34.0 |
| | SD | 1.13 | 2.17 | 5.72 | 28.8 | 20.4 |
| | CV % | 58.2 | 31.0 | 36.2 | 76.3 | 60.0 |
| Cₐᵥ (ng/mL) | N | 6 | 6 | 6 | 6 | 4 |
| | Mean | 3.39 | 12.5 | 26.6 | 60.0 | 60.1 |
| | SD | 1.67 | 4.56 | 9.71 | 34. | 30.4 |
| | CV % | 49.4 | 36.6 | 36.5 | 57.4 | 50.5 |
| CL/F | N | 4 | 6 | 6 | 6 | 4 |
| (L/h) | Mean | 125 | 66.6 | 63.1 | 45.9 | 62.9 |
| | SD | 66.4 | 22.2 | 23.4 | 19.0 | 37.1 |
| | CV % | 52.9 | 33.4 | 37.0 | 41.4 | 59.0 |
| R_{acc,Cmax} | N | 6 | 6 | 6 | 6 | 4 |
| | Mean | 1.55 | 1.78 | 1.97 | 3.37 | 2.79 |
| | SD | 0.408 | 0.571 | 0.636 | 1.02 | 0.751 |
| | CV % | 26.4 | 32.0 | 32.3 | 30.3 | 26.9 |
| R_{acc,AUC} | N | 6 | 6 | 6 | 6 | 4 |
| | Mean | 1.75 | 2.17 | 2.61 | 3.79 | 3.25 |
| | SD | 0.300 | 0.575 | 0.719 | 1.01 | 1.14 |
| | CV % | 17.2 | 26.5 | 27.5 | 26.7 | 35.2 |

Prolactin increased in a dose-and concentration-dependent manner, with greater increase seen after first dose.

Three subjects discontinued due to adverse events. Majority of the treatment emergent adverse events (TEAEs) were mild to moderate. Most common were headache, somnolence, dizziness, dizziness postural, micturition urgency & orthostatic hypotension. Five TEAEs occurring in 3 subjects (all after cessation of Study Drug dosing) were severe. One serious adverse event was reported after stopping treatment with Study Drug 24 mg TID, consistent with abrupt discontinuation of serotonergic drugs. This subject accounted for 3 severe TEAEs. The data support further development in developmental & epileptic encephalopathies and other seizure disorders.

In sum, Study Drug was rapidly absorbed into systemic circulation following administration of first dose and after multiple oral dose administrations of Study Drug powder-in-capsule formulation. The peak plasma concentrations after the first and last dose occurred at a median Tₘₐₓ between 1.00 and 2.00 hours.

Most subjects were at steady state after 10 days of dosing in non-titration part Multiple dosing with a TID regimen resulted in dose dependent accumulation.

Accumulation ratio for AUCₜₐᵤ increased from 1.75 to 3.79 from 3 to 18 mg in Part C and was 3.25 for 12/24 mg dose titration regimen. Accumulation ratio for Cₘₐₓ increased from 1.55 to 3.37 from 3 to 18 mg in Part C and was slightly less at 2.79 with 12/24 mg dose titration regimen.

Study Drug mean half-life at steady state was consistent among all dose groups and ranged from 4.81 to 6.50 hours.

At steady state, 20.9% to 32.3% of the dose was eliminated by the renal route in 48 hours across doses of 3 to 18 mg TID dosing regimen. In the 12/24 mg TID titration regimen, only 12.3% of the dose was eliminated by the renal route in 48 hours.

The mean percent change from baseline prolactin level at 2 hours increased with increasing doses from 3 mg to 12 mg and plateaued thereafter. The data were highly variable.

### Example 3

A Phase 1b/2a randomized, double-blind, placebo-controlled, parallel-group, dose-escalation study will be conducted to investigate the safety, tolerability, pharmacokinetics, pharmacodynamics, and exploratory efficacy of Study Drug in subjects with developmental and epileptic encephalopathies.

The objectives and endpoints for the study may include the following:

| OBJECTIVES | ENDPOINTS |
|---|---|
| Primary Objective: | Primary Endpoints: |
| To investigate the safety and | Incidence and severity of treatment-emergent |
| tolerability of multiple doses | adverse events (TEAEs), including serious |
| of Study Drug in adult | adverse events (SAEs) and adverse events |
| subjects with developmental | (AEs) leading to discontinuation |
| and epileptic | Safety laboratory parameters |
| encephalopathies. | Physical examination findings |
| | Vital signs |
| | Body weight |
| | 12-lead electrocardiograms (ECGs) |
| | Columbia-Suicide Severity Rating Scale (C- |
| | SSRS) responses |
| | Patient Health Questionnaire-9 (PHQ-9) total |
| | score and Item 9 score |
| Secondary Objectives: | Secondary Endpoints: |
| To characterize the | Observed concentrations by time and dose, |
| pharmacokinetics (PK) of | modeled plasma concentration-time profile, |
| Study Drug in adults with | modeled population estimates of apparent |
| developmental and epileptic | clearance (CL/F), apparent volume of |
| encephalopathies. | distribution (V/F), inter- and intra-individual |
| | variability in PK parameters, and covariate |
| | analysis |
| To characterize the | Observed and change from baseline in serum |
| pharmacodynamic (PD) | prolactin concentrations by dose, visit, and |
| effects of Study Drug on | nominal time point |
| prolactin. | |
| To evaluate and characterize | Model estimated PK parameters, for example, |
| PK-PD relationships of | but not limited to, average plasma |
| Study Drug for endpoints | concentration (C_{avg}), observed concentration |
| related to safety and | just prior to dosing (C_{trough}) and PD (ie, |
| seizures. | prolactin), safety endpoints of interest (eg, |
| | AEs, clinical laboratory tests [aspartate |
| | aminotransferase/alanine aminotransferase], |
| | vital signs [eg, heart rate, blood pressure]), |
| | and seizure counts |
| To identify the optimal | Simulated dose-response curve |
| dose(s) of Study Drug for | |
| Phase 2 clinical studies. | |
| Exploratory Objectives: | Exploratory Endpoints: |
| To investigate the effect of | Percentage change from baseline in observed |
| Study Drug on the frequency | countable motor seizure frequency during the |
| of observed | treatment period. |
| convulsive/motor seizures | Percent of subjects with ≥50% reduction in |
| and other seizure types. | total seizures during the treatment period. |
| | Percent change from baseline in non- |
| | convulsive seizure frequency. |
| | Percent change from baseline in the number |
| | of episodes of status epilepticus during |
| | treatment. |
| | Percent of subjects requiring rescue |
| | medication during treatment. |
| | Percent of subjects with countable motor |
| | seizure-free days during treatment. |
| | Lennox-Gastaut Syndrome: Percentage |
| | change in observed drop seizure frequency |
| | during the treatment period. |
| To investigate the effect of | Percent of subjects with at least a 1-point |
| Study Drug on the | change from baseline in CGI-I/CGI-S during |
| Subject/Caregiver and | treatment. |
| Investigator Clinical Global | Evaluation of change in quality of life using |
| Impression of Improvement | the QOLCE-55. |
| (CGI-I), the Investigator | |
| Clinical Global Impression | |
| of Severity (CGI-S), and the | |
| 55-item Quality of Life in | |
| Childhood Epilepsy | |
| Questionnaire (QOLCE-55). | |
| To evaluate the relationships | Graphical exploration of genotypic variants |
| between genetic | for metabolic enzymes and transporters and |
| polymorphisms in drug | exposure endpoints (e.g., observed |
| metabolizing enzymes, | concentration just prior to dosing, model |
| transporters, and Study Drug | estimated average plasma concentration.) |
| exposure. | |

The study consists of the following periods:
Screening (7 days)/Baseline (28 days)
Part 1—randomization and up-titration (15 days)
Part 2—maintenance (60 days)
Part 3—down-titration/taper (up to 15 days)
Follow-up (30 days after completion of down-titration)

The target highest dose of 12 mg TID will be reached after a 15-day up-titration period, if tolerated. The double-blind treatment period will consist of the Part 1 randomization and up-titration (15 days) and the Part 2 maintenance (60 days).

### Part 1: Up-Titration (Days 1 to 15)

During the up-titration period, subjects will be titrated up to 12 mg TID or highest tolerated dose. The planned Study Drug or placebo dose-escalation steps in the up-titration period are 6 mg TID (Days 1 to 5), 9 mg TID (Days 6 to 10), and 12 mg TID (Days 11 to 15) or matching placebo TID. Subjects who cannot tolerate up-titration to 12 mg TID may have their dose reduced to 9 mg TID. Subjects who cannot tolerate up-titration to 9 mg TID may have their dose reduced to 6 mg TID.

### Part 2: Maintenance (Days 16 to 75)

After completing up-titration to a dose of either 6 mg TID, 9 mg TID, or 12 mg TID Study Drug or placebo TID, subjects will continue at these dose levels for the 60-day maintenance period. During the maintenance period, if a subject cannot tolerate their assigned dose level from the up-titration period, they will be discontinued.

### Part 3: Down-Titration/Taper (Days 76 to Day 80/85/90)

During the down-titration/taper period, study drug will be reduced in increments of 3 mg TID every 5 days until the subject is no longer taking Study Drug or placebo as follows:

### 12-mg Dose: 15-Day Down-Titration/Taper Period

Days 76 to 80 (first 5 days of taper)=9 mg Study Drug TID or placebo TID
Days 81 to 85 (second 5 days of taper)=6 mg Study Drug TID or placebo TID
Days 86 to 90 (third 5 days of taper)=3 mg TID or placebo TID

### 9-mg Dose: 10-Day Down-Titration/Taper Period

Days 76 to 80 (first 5 days of taper)=6 mg Study Drug TID or placebo
Days 81 to 85 (second 5 days of taper)=3 mg Study Drug TID or placebo TID

### 6-mg Dose: 5-Day Down-Titration/Taper Period

Days 76 to 80 =3 mg Study Drug TID or placebo TID

The total daily dose will not exceed 36 mg.

Subjects who meet all of the following inclusion criteria and none of the following exclusion criteria will be eligible to participate in this study.

Inclusion criteria may include:
Male or female adults >18 to <65 years of age at the time of screening with a body mass index (BMI) <35 kg/m2 and >18 kg/m2.
Diagnosis of DEE that includes Dravet syndrome, Lennox-Gastaut Syndrome, and other DEEs and demonstrates treatment-resistant countable motor seizures with an average of ≥4 observed/countable motor seizures per 4-week period during the 12 weeks before screening while on stable anti-seizure medicine (ASM) treatment based on the subject/caregiver report and investigator's assessment.
   a. Subjects who are characterized as having a DEE but who do not have Dravet syndrome or Lennox-Gastaut Syndrome will have all of the following based on the subject/caregiver history and investigator's assessment.
      i. History of onset of unprovoked seizures at 5 years of age or earlier.
      ii. History of developmental delay.
      iii. History of combined focal and generalized seizure types or multiple generalized seizure types.
      iv. History of slow or disorganized electroencephalogram.
      v. No history of idiopathic generalized seizures.
      vi. Had an average of >4 observed countable motor seizures per 4-week period during the 12 weeks before screening while on stable ASM treatment based on the subject/caregiver report and investigator's assessment.
   b. Subjects characterized as having Dravet syndrome must meet all the following:
      i. Onset of seizures between 3 and 12 months of age in an otherwise healthy infant.
      ii. History of seizures that were either generalized tonic-clonic or unilateral clonic or bilateral clonic.
      iii. Initial development was normal.
      iv. History of developmental delay.
      v. Lack of alternative diagnosis.
      vi. Had an average of >4 observed countable motor seizures per 4-week period during the 12 weeks before screening while on stable ASM treatment based on the subject/caregiver report and investigator's assessment.
AND at least 1 of the following:
   vii. Emergence of another seizure type, including myoclonic, generalized tonic-clonic, tonic, atonic, absence, and/or focal developed after the first seizure type.
   viii. Prolonged exposure to warm temperatures-induced seizures and/or seizures that were associated with fevers due to illness or vaccines, hot baths, high levels of activity, and sudden temperature changes, and/or seizures were induced by strong natural and/or fluorescent lighting, as well as certain visual patterns.
   ix. Genetic test results consistent with a diagnosis of Dravet syndrome (pathogenic, likely pathogenic, variant of unknown significance, or inconclusive but unlikely to support an alternative diagnosis).
c. Subjects characterized as having Lennox-Gastaut Syndrome must meet the following:
   i. History of tonic seizures or tonic/atonic seizures.
   ii. More than 1 type of generalized seizure, including but not limited to generalized tonic-clonic, tonic-atonic, atonic, tonic, myoclonic, or DROP SEIZURES for ≥6 months before screening. Drop seizures are defined as a seizure involving the entire body, trunk, or head that leads to a fall, injury, slumping in a chair, or head hitting the surface, or could have led to a fall or injury, depending on the position of the subject at the time of the attack or spell.
   iii. A history of seizure before 8 years of age.
   iv. History of developmental delay.
   v. Documentation of previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity).
   vi. Had an average of >4 observed drop seizures per 4-week period during the 12 weeks before screening while on stable ASM treatment based on the subject/caregiver report and investigator's assessment.
subject's legally acceptable representative is willing to keep the regimen(s) stable throughout the study.
All medications or interventions for epilepsy (including ketogenic diet and vagus nerve stimulation) must have been stable for 4 weeks before screening and are expected to remain stable throughout the study.
G-tubes/PEG tubes (where present) should have been placed and been functioning for at least 3 months before screening. Naso-gastric tubes are not allowed.

Randomization inclusion criteria may include:
Had a stable baseline with ≥4 observed countable motor seizures during the 28-day Baseline period, with a minimum of 2 in the first 14 days and 2 in the second 14 days and was not seizure-free for more than 21 consecutive days during the 28-day Baseline period.
The subject, parent, or caregiver has been compliant with diary completion during the Baseline period, in the opinion of the investigator (i.e., at least 80% compliant but not more than 5 total days missing).
The subject, parent, or caregiver responsible for study drug administration demonstrates an understanding of how to administer study drug correctly

Subjects may be excluded from the study if any of the following criteria apply:
Has been admitted to a medical facility for treatment of status epilepticus requiring mechanical respiration within 3 months before screening.
Current or prior idiopathic generalized seizures.
Has had a vagus nerve stimulator implanted within 6 months before screening, and/or settings have been changed within 1 month before screening and/or anticipated to change during the study.
Is on a ketogenic diet that has been started within 6 months before the Screening Visit, has been changed within 1 month of the Screening Visit, or is anticipated to change during the study.
Subjects with Dravet syndrome who have used sodium channel blockers, phenytoin, fosphenytoin, carbamazepine, oxcarbazepine, eslicarbazepine, lamotrigine, lacosamide, rufinamide, vigabatrin, tiagabine, pregabalin, and gabapentin within the last month (30 days) before screening.
Current use of felbamate, unless the subject is on a stable dose of felbamate ≥12 months before screening with stable liver function and hematology laboratory tests.
Current use of topiramate, unless the subject is on a stable dose of topiramate ≥12 months before screening with stable weight.
Has used fenfluramine.
Has a clear pattern of perimenstrual exacerbation of seizures.
Considered at risk of suicidal behavior based on the C-SSRS. A subject should be excluded, and a risk assessment should be done by a qualified mental health professional (MHP) if the subject has had suicidal intent (Ideation items 4 or 5) in the last 6 months before randomization, or suicidal behaviors or attempts in the past year. If the subject is unable to complete the C-SSRS due to developmental status, an authorized representative may be used for the completion of the C-SSRS. The investigator may also use clinical judgment, which must then be documented in the source document.
PHQ-9 score of >9 or a positive response to Question 9.
Current or recent history of moderate or severe depression, anorexia nervosa, or bulimia per the Diagnostic and Statistical Manual of Mental Disorders, Fifth Edition, within the prior year.
Positive for human immunodeficiency virus (HIV), hepatitis B, or hepatitis C infections. Note that subjects who have been vaccinated against hepatitis B (hepatitis B surface antibody-positive) who are negative for other markers of prior hepatitis B infection (e.g., negative for hepatitis B core antibody) are eligible. Also note that subjects who are positive for hepatitis C antibody are eligible as long as they have a negative hepatitis C viral load by quantitative polymerase chain reaction.
Has an abnormal and clinically significant 12-lead ECG at screening in the opinion of the investigator, for example, second-or third-degree heart block or a corrected QT interval (QTc) of >450 msec for males or >470 msec for females. Entry of any subject with an abnormal but not clinically significant ECG must be approved and documented by signature by the investigator or appropriately qualified delegate.
Has a supine or sitting blood pressure >140 mm Hg systolic or 90 mm Hg diastolic or <90 mm Hg systolic or 60 mm Hg diastolic.
Has evidence or a history of orthostatic hypotension.
Has a sustained heart rate increase of >30 beats/minute within 3 minutes of standing from a supine or sitting position.
History of progressive neurodegeneration indicated by magnetic resonance imaging.
Has moderate or severe renal impairment.
History of glaucoma.
Past history of significant lower urinary tract pathology, including bladder outflow obstruction, urine retention, urethral stricture, bladder neck contracture, bladder diverticulum, or bladder stone.
Past history of recurrent urinary tract infections (UTIs) (≥2 documented UTIs in the past 12 months) or current evidence of UTI.
Has abnormal clinical laboratory test results at screening that suggest a clinically significant underlying hepatic disease. If the subject has alanine aminotransferase (ALT) and/or aspartate aminotransferase (AST)>2.5×the upper limit of normal (ULN) and/or elevated bilirubin>2×ULN, then the Medical Monitor should be consulted.
Receiving concomitant therapy with centrally-acting anorectic agents; monoamine oxidase inhibitors; any centrally-acting compound with a clinically appreciable amount of serotonin agonist or antagonist properties, including serotonin and norepinephrine reuptake inhibitors, serotonin reuptake inhibitors; atomoxetine, vortioxetine, or other centrally-acting noradrenergic agonists or agents intended to facilitate weight loss.
Use of narrow therapeutic index substrates of CYP1A2 during the study.
Concomitant use of strong or moderate inhibitors of CYP1A2, and other disallowed drugs/drug classes.
Concomitant use of drugs whose exposures might be altered by Study Drug, from first dose of study drug until follow-up. Refer to the most recent Investigator's Brochure for guidance.
Use of grapefruit juice, grapefruit, Seville oranges or St John's Wort or products containing these within 30 days before the first administration of study drug, and until the follow-up visit.
Use of any cannabidiol products except Epidiolex.
Has a positive result on the urine drug screen, including tetrahydrocannabinol, but excluding other controlled medications taken as prescribed.
Unstable, clinically significant neurologic (other than the disease being studied), psychiatric, cardiovascular, pulmonary, hepatic, renal, metabolic, gastrointestinal, urologic, immunologic, hematopoietic, or endocrine disease or other abnormality which may impact the ability of the subject to participate or potentially confound the study results.
Has pulmonary arterial hypertension, current or past history of cardiovascular or cerebrovascular disease, such as cardiac valvulopathy, myocardial infarction, or stroke.

Study Drug and placebo doses should be administered TID and flushed each time with 120 mL of non-carbonated water. Doses may be given without regard to food. Subjects who are following a ketogenic diet should maintain stable dietary habits and the diet should not be altered during the study unless medically necessary and documented. For subjects receiving study drug via G-tube/PEG tube, other medications or enteral feeds should not be given concurrently.

It is recommended that subjects should stay upright (seated, standing or ambulatory) for approximately 1 hour after study drug dosing on the PK sampling days, if possible.

The following medications and products are excluded from the Screening Visit (Visit 1) until the end of the Follow-up Visit (Visit 10):
Fenfluramine
Lorcaserin
Drugs that can worsen seizures in subjects with known or suspected Dravet syndrome e.g., sodium channel blockers, phenytoin, fosphenytoin, carbamazepine, oxcarbazepine, eslicarbazepine, lamotrigine, lacosamide, rufinamide, vigabatrin, tiagabine, pregabalin, and gabapentin.
Centrally-acting anorectic agents; monoamine oxidase inhibitors; any centrally-acting compound with clinically appreciable amount of serotonin agonist or antagonist properties, including serotonin and norepinephrine reuptake inhibitors, serotonin reuptake inhibitors; atomoxetine, vortioxetine, or other centrally-acting noradrenergic agonists or agents intended to facilitate weight loss.

Subjects should be advised to not consume alcohol or drugs such as cannabis during the study. Subjects must be instructed not to take any medications, including over-the-counter products, without first consulting with the investigator.

Study Drug or placebo will be administered as a liquid either orally or through a G-tube/PEG tube. Complete instructions will be provided to the subjects/caregivers in a separate document.

Each subject and/or caregiver will be instructed to administer the study drug, orally or via G-tube/PEG tube, TID and flushed each time with 120 mL of non-carbonated water. The 3 daily doses should be administered at least 6 hours apart. For example, a suggested dosing schedule is to administer the morning dose upon waking and no later than 8 AM, the afternoon dose at approximately 3 PM, and the night dose at bedtime at 10 PM or later. Doses may be given with or without food. For subjects receiving study drug via G-tube/PEG tube, other medications, or enteral feeds should not be given concurrently.

### Efficacy Assessments

### Seizure Frequency

Seizure frequency will be quantified as an exploratory measure of the antiseizure activity of Study Drug versus placebo. When utilized as a continuous variable, seizure frequency is considered a sensitive measure of efficacy.

In the pre-treatment phase, existing therapy will remain stable and baseline seizure frequency and seizure type will be recorded in a diary over 28 days over each 24-hour day.

During the double-blind treatment period (Parts 1 and 2), seizure frequency and seizure type will be recorded in an electronic diary (e-diary) over the entire 75-day double-blind treatment period over each 24-hour day.

Only the primary caregiver or the subject, not both, throughout the study may enter seizure information into the e-diary. It is expected that e-diaries will be completed at the beginning of each day accounting for the day before; however, when needed, seizure diary entries may be completed up to 3 days after seizures occurred.

### Clinical Global Impression of Improvement

The overall level of improvement due to treatment will be assessed using the Clinical Global Impression of Improvement (CGI-I). This scale will be completed by the parent/caregiver and by the investigator at visits outlined in the SoA. The CGI-I 7-point scale is as follows: “Very Much Improved” (1); “Much Improved” (2); “Slightly Improved” (3); “No Change” (4); “Slightly Worse” (5); “Much Worse” (6); “Very Much Worse” (7). Whenever possible, the same rater should complete their respective scales throughout the study.

### Clinical Global Impression of Severity

The Clinical Global Impression of Severity (CGI-S) will be assessed by the investigator at visits outlined in the SoA. The CGI-S asks the clinician the following question: Considering your total clinical experience with this particular population, how ill is the patient at this time?” which is rated on the following 7-point scale: 1=normal, not at all ill; 2=borderline ill; 3=mildly ill; 4=moderately ill; 5=markedly ill; 6=severely ill; 7=among the most extremely ill patients. Whenever possible, the same rater should complete their respective scales throughout the study.

### Quality of Life in Childhood Epilepsy Questionnaire

The Quality of Life in Childhood Epilepsy Questionnaire-55 (QOLCE-55) is a 55-item questionnaire that measures the impact of epilepsy on overall life functioning and well-being in children; however, the scale has been used in young adults with DEE-related cognitive impairment who may not be able to complete a patient-reported quality of life scale. The QOLCE-55 includes 4 domains: cognitive functioning (22 items), emotional functioning (17 items), social functioning (7 items), and physical functioning (9 items). The questionnaire should be completed by the subject's parent/caregiver. Higher scores indicate better quality of life and/or increased well-being. Whenever possible, the same rater should complete their respective scales throughout the study.

Although the disclosure has been described with reference to the above examples, it will be understood that modifications and variations are encompassed within the spirit and scope of the disclosure. The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1-3. (canceled)

4. A method for treating a seizure disorder comprising: prescribing and/or administering to an individual in need thereof, (R)-N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt, hydrate, or solvate thereof, wherein Compound 1, or a pharmaceutically acceptable salt, hydrate, or solvate thereof, is administered via a titration scheme that comprises the up-titration of Compound 1, or a pharmaceutically acceptable salt, hydrate, or solvate thereof, until an optimized dose is administered.

5. The method of claim 4, wherein the seizure disorder is selected from epilepsy, epilepsy with generalized tonic-clonic seizures, epilepsy with myoclonic absences, frontal lobe epilepsy, temporal lobe epilepsy, Landau-Kleffher Syndrome, Rasmussen's syndrome, Dravet syndrome, Doose syndrome, CDKL5 disorder, infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome, CDKL5 disorder, childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

6-10. (canceled)

11. The method of any one of the preceding claims claim 4, wherein said administration results in an improvement in the frequency of convulsive/motor seizures and other seizure types.

12. The method of claim 11, wherein said administration results in an improvement in one or more of the following:
frequency of observed countable motor seizures;
number of total seizures;
frequency of non-convulsive seizure;
number of episodes of status epilepticus;
frequency of use of rescue medication; and/or
number of countable motor seizure-free days.

13. The method of claim 4, wherein said administration results in an improvement in the Subject/Caregiver and Investigator Clinical Global Impression of Improvement (CGI-I), the Investigator Clinical Global Impression of Severity (CGI-S), and/or the 55-item Quality of Life in Childhood Epilepsy Questionnaire (QOLCE-55).

14. The method of claim 13, wherein said administration results in at least a 1-point change from baseline in CGI-I and/or CGI-S.

15. The method of claim 4, wherein prior to administration, the individual had treatment resistant countable motor seizures with an average of ≥4 observed/countable motor seizures per 4-week period while on stable ASM treatment.

16. The method of claim 4, wherein the seizure disorder is a DEE but is not Dravet syndrome or Lennox-Gastaut Syndrome.

17. The method of claim 16, wherein prior to administration, the individual had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

18. The method of claim 4, wherein the seizure disorder is Dravet syndrome.

19. The method of claim 18, wherein prior to administration, the individual had:
onset of seizures between 3 and 12 months of age in an otherwise healthy infant; a history of seizures that were either generalized tonic-clonic or unilateral clonic or bilateral clonic;
normal initial development; and/or
a history of developmental delay.

20. The method of claim 18, wherein prior to administration, the individual had:
an emergence of another seizure type;
prolonged exposure to warm temperatures-induced seizures and/or seizures that were associated with fevers due to illness or vaccines, hot baths, high levels of activity, and sudden temperature changes, and/or
seizures were induced by strong natural and/or fluorescent lighting.

21. The method of claim 18, wherein prior to administration, the individual had genetic test results consistent with a diagnosis of Dravet syndrome.

22. The method of claim 4, wherein the seizure disorder is Lennox-Gastaut Syndrome.

23. The method of claim 22, wherein prior to administration, the individual had:
a history of tonic seizures or tonic/atonic seizures;
more than 1 type of generalized seizure, including but not limited to generalized tonic-clonic, tonic-atonic, atonic, tonic, myoclonic, or drop seizures;
a history of seizure before 8 years of age;
a history of developmental delay;
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

24. The method of claim 4, wherein the titration scheme comprises prescribing and/or administering Compound 1, or a pharmaceutically acceptable salt, hydrate, or solvate thereof, at an initial dose equivalent to 6 mg of Compound 1 three time daily for about five days and, provided that the individual tolerates the initial dose and that the individual has not had an adequate response, increasing the dose.

25. The method of claim 24, wherein the increased dose is equivalent to 9 mg of Compound 1 three time daily.

26. The method of claim 24, wherein the titration scheme further comprises administering Compound 1, or a pharmaceutically acceptable salt, hydrate, or solvate thereof, at the increased dose for about five days.

27. The method of claim 24, wherein if the individual does not tolerate the increased dose, the optimized dose is the initial dose.

28. The method of claim 24, wherein if the individual tolerates the increased dose and if the individual has had an adequate response, the optimized dose is the increased dose.

29. The method of claim 27, further comprising administering the optimized dose of Compound 1, or a pharmaceutically acceptable salt, hydrate, or solvate thereof, to the individual.

30. The method of claim 26, wherein if the individual tolerates the increased dose and if the individual has not had an adequate response, the method further comprises increasing the dose.

31. The method of claim 30, wherein the further increased dose is equivalent to about 12 mg of Compound 1 three times daily.

32. The method of claim 30, wherein if the individual does not tolerate the further increased dose, the optimized dose is the increased dose.

33. The method of claim 30, wherein if the individual tolerates the further increased dose and if the individual has had an adequate response, the optimized dose is the further increased dose.

34. The method of claim 32, further comprising administering the optimized dose of Compound 1, or a pharmaceutically acceptable salt, hydrate, or solvate thereof, to the individual.

35. The method of claim 4, wherein the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

36. The method of claim 4, further comprising down-titrating the Compound 1, or a pharmaceutically acceptable salt, hydrate, or solvate thereof.

37. The method of claim 4, wherein the Compound 1, or a pharmaceutically acceptable salt, hydrate, or solvate thereof, is an HCl salt of Compound 1.
